(19) United States
(12) Patent Application Publication
(10) Pub. No.: US 2019/0029546 A1
(43) Pub. Date: Jan. 31, 2019

(54) Arteriosclerosis Measurement Instrument and Arteriosclerosis Measurement Method

(71) Applicant: Lohas Tech Co., Ltd, Beijing (CN)

(72) Inventors: Chi CHEN, Beijing (CN); Yao WANG, Beijing (CN)

(21) Appl. No.: 15/841,334

(22) Filed: Dec. 14, 2017

(30) Foreign Application Priority Data
Jul. 27, 2017 (CN) 201710628734.8

(51) Int. Cl. A61B 5/024; A61B 5/00; A61B 5/02; A61B 5/0255

(52) U.S. Cl. CPC A61B 5/02444; A61B 5/6824; A61B 5/6829; A61B 5/02007; A61B 5/7246; A61B 5/0255; A61B 5/742; A61B 5/6833; A61B 5/02438; A61B 5/6834; A61B 5/6831; A61B 5/7278

(57) ABSTRACT

Disclosed are an arteriosclerosis measurement instrument, wherein the measurement instrument comprises: an upper arm vibration sensor component attached onto a skin surface of a to-be-measured upper arm through a first attachment, wherein the upper arm vibration sensor component is configured to collect an upper-arm pulse vibration signal of the to-be-measured upper arm; an ankle vibration sensor component attached onto a skin surface of a to-be-measured ankle through a second attachment, wherein the ankle vibration sensor component is configured to collect an ankle pulse vibration signal of the to-be-measured ankle; and a micro-processor, configured to determine a time difference between the pulse vibration signal of a tested person according to the upper-arm pulse vibration signal collected by the upper arm vibration sensor component and the ankle pulse vibration signal collected by the ankle vibration sensor component, and configured to obtain an arteriosclerosis measurement result according to the determined transmission time difference.

### TECHNICAL FIELD

The present disclosure relates to the field of signal processing, and in particular to an arteriosclerosis measurement instrument and an arteriosclerosis measurement method.

### BACKGROUND

Brachial-ankle Pulse Wave Velocity (baPWV) is an arteriosclerosis measurement index that has been widely acknowledged. In relevant arts, all the measurements for baPWV are to employ a cuff device to measure the pulse waves of arms and ankles respectively, and, through the measurement of the pulse waves of arms and ankles, an arteriosclerosis measurement result is determined.

However, when a cuff device is employed to measure the pulse waves of arms and ankles, the cuff needs to be tightly bound onto the arms and ankles; that is to say, measurement of pulse waves is achieved by squeezing arm and ankles to an almost unbearable level.

Because the inflation of cuff will lead to squeezing arms or ankles, some patients with particular health conditions are not suitable for an inflatable cuff device. For example, when an inflatable cuff device is employed to perform detection, internal haemorrhage may be caused due to the long-term compression onto the upper arms and ankles. Moreover, patients are not suitable for wearing a cuff in the following conditions: the patients are having an intravenous drip on the upper arms, and the patients are blood shunting on the upper arms when accepting hematodialysis. In addition, when the body parts wearing the cuff have acute inflammation, purulent diseases, injuries and the like, the symptoms will get worse if the patients wear the cuff. Furthermore, healthy people are still likely to feel nervous and uncomfortable after using the inflatable cuff devices, sometimes even have pressure injuries.

Therefore, in relevant art, there is a problem of insufficient universality when an inflatable cuff device is employed to measure arteriosclerosis.

In-view of the above problems, no effective solution has been proposed so far.

### SUMMARY

The embodiment of the present disclosure provides an arteriosclerosis measurement instrument and an arteriosclerosis measurement method, to at least solve the problem of insufficient universality when an inflatable cuff device is employed to measure arteriosclerosis in relevant art.

According to one aspect of the embodiment of the present disclosure, an arteriosclerosis measurement instrument is provided, including: an upper arm vibration sensor component attached onto a skin surface of a to-be-measured upper arm through a first attachment, wherein the upper arm vibration sensor component is configured to collect an upper-arm pulse vibration signal of the to-be-measured upper arm; an ankle vibration sensor component attached onto a skin surface of a to-be-measured ankle through a second attachment, wherein the ankle vibration sensor component is configured to collect an ankle pulse vibration signal of the to-be-measured ankle; and a micro-processor, configured to determine a time difference between the above pulse vibration signals of a tested person according to the upper-arm pulse vibration signal collected by the upper arm vibration sensor component and the ankle pulse vibration signal collected by the ankle vibration sensor component, and configured to obtain an arteriosclerosis measurement result according to the determined transmission time difference.

According to an embodiment of the present disclosure, the upper arm vibration sensor component includes: a left upper arm vibration sensor component attached onto a skin surface of a to-be-measured left upper arm through a first sub-attachment, and/or a right upper arm vibration sensor component attached onto a skin surface of a to-be-measured right upper arm through a second sub-attachment, wherein the first attachment includes the first sub-attachment and/or the second sub-attachment; and the ankle vibration sensor component includes: a left ankle vibration sensor component attached onto a skin surface of a to-be-measured left ankle through a third sub-attachment, and/or a right ankle vibration sensor component attached onto a skin surface of a to-be-measured right ankle through a fourth sub-attachment, wherein the second attachment includes the third sub-attachment and/or the fourth sub-attachment.

According to an embodiment of the present disclosure, the arteriosclerosis measurement instrument further including: a display, configured to display at least one of following: the upper-arm pulse vibration signal collected by the upper arm vibration sensor component, the ankle pulse vibration signal collected by the ankle vibration sensor component and the determined arteriosclerosis measurement result.

According to an embodiment of the present disclosure, the first attachment is a sticking assembly or an adsorbing assembly, and/or, the second attachment is a sticking assembly or an adsorbing assembly.

According to an embodiment of the present disclosure, the sticking assembly is a double faced adhesive tape, and/or, the adsorbing assembly is a suction cup.

According to an aspect of the present disclosure, an arteriosclerosis measurement method is provided, including: attaching an upper arm vibration sensor component onto a skin surface of a to-be-measured upper arm through a first attachment; collecting an upper-arm pulse vibration signal of the to-be-measured upper arm through the upper arm vibration sensor component; attaching an ankle vibration sensor component onto a skin surface of a to-be-measured ankle through a second attachment; collecting an ankle pulse vibration signal of the to-be-measured ankle through the ankle vibration sensor component; determining a time difference between the above pulse vibration signals of a tested person according to the upper-arm pulse vibration signal collected by the upper arm vibration sensor component and the ankle pulse vibration signal collected by the ankle vibration sensor component, and obtaining an arteriosclerosis measurement result according to the determined transmission time difference.

According to an embodiment of the present disclosure, attaching the upper arm vibration sensor component onto the skin surface of the to-be-measured upper arm through the first attachment includes: attaching a left upper arm vibration sensor component onto a skin surface of a to-be-measured left upper arm through a first sub-attachment, and/or attaching a right upper arm vibration sensor component onto a skin surface of a to-be-measured right upper arm through a second sub-attachment, wherein the first attachment includes the first sub-attachment and/or the second sub-attachment, the upper arm vibration sensor component includes the left upper arm vibration sensor component and/or the right upper arm vibration sensor component; and attaching the ankle vibration sensor component onto the skin surface of the to-be-measured ankle through the second attachment includes: attaching a left ankle vibration sensor component onto a skin surface of a to-be-measured left ankle through a third sub-attachment, and/or attaching a right ankle vibration sensor component onto a skin surface of a to-be-measured right ankle through a fourth sub-attachment, wherein the second attachment includes the third sub-attachment and/or the fourth sub-attachment, and the ankle vibration sensor component includes the left ankle vibration sensor component and/or the right ankle vibration sensor component.

According to an embodiment of the present disclosure, attaching the upper arm vibration sensor component onto the skin surface of the to-be-measured upper arm through the first attachment includes: sensing an upper arm pulse sensitive part of the skin surface of the to-be-measured upper arm, and attaching the upper arm vibration sensor component onto the upper arm pulse sensitive part of the skin surface of the to-be-measured upper arm through the first attachment; and/or, attaching the ankle vibration sensor component onto the skin surface of the to-be-measured ankle through the second attachment includes; sensing an ankle pulse sensitive part of the skin surface of the to-be-measured ankle, and attaching the ankle vibration sensor component onto the ankle pulse sensitive part of the skin surface of the to-be-measured ankle through the second attachment.

According to an embodiment of the present disclosure, after collecting the upper-arm pulse vibration signal of the to-be-measured upper arm through the upper arm vibration sensor component, and/or, collecting the ankle pulse vibration signal of the to-be-measured ankle through the ankle vibration sensor component, the method further includes: displaying the collected upper-arm pulse vibration signal and the collected ankle pulse vibration signal; or, after obtaining the arteriosclerosis measurement result according to the determined transmission time difference, the method further includes: displaying the determined arteriosclerosis measurement result.

According to an embodiment of the present disclosure, the first attachment is a sticking assembly or an adsorbing assembly, and/or, the second attachment is a sticking assembly or an adsorbing assembly.

According to an embodiment of the present disclosure, the sticking assembly is a double faced adhesive tape, and/or, the adsorbing assembly is a suction cup.

In the embodiment of the present disclosure, a vibration sensor component (which, for example, may include: an upper arm vibration sensor component and an ankle vibration sensor component) is attached, by an attachment, onto a skin surface to be measured, a pulse vibration signal is collected through the vibration sensor component, and an arteriosclerosis measurement result is obtained according to the collected pulse vibration signal; in this way, the arteriosclerosis measurement result is successfully obtained in a condition of not compressing the skin surface to be measured; thus, the technical problem of insufficient universality when an inflatable cuff device is employed to measure arteriosclerosis is solved.

### BRIEF DESCRIPTION OF THE DRAWINGS

For a better understanding of the present disclosure, accompanying drawings described hereinafter are provided to constitute one part of the application; the schematic embodiments of the present disclosure and the description thereof are used to illustrate the present disclosure but to limit the present disclosure improperly. In the accompanying drawings:

FIG. 1 is a structural schematic diagram of an arteriosclerosis measurement instrument according to the embodiment of the present disclosure.

FIG. 2 is a preferred structural schematic diagram (1) of an arteriosclerosis measurement instrument according to the embodiment of the present disclosure.

FIG. 3 is a preferred structure block diagram (2) of an arteriosclerosis measurement instrument according to the embodiment of the present disclosure.

FIG. 4 is a flowchart of an arteriosclerosis measurement method according to the embodiment of the present disclosure.

FIG. 5 is a structure block diagram of an arteriosclerosis measurement device according to the embodiment of the present disclosure.

### DETAILED DESCRIPTION OF THE EMBODIMENTS

To make those skilled in the art better understand the scheme of the present disclosure, a clear and complete description as below is provided to the technical scheme in the embodiments of the present disclosure in conjunction with the accompanying drawings in the embodiments of the present disclosure. Obviously, the embodiments described hereinafter are simply part embodiments of the present disclosure, but all the embodiments. All other embodiments obtained by the ordinary skill in the art based on the embodiments in the present disclosure without creative work are intended to be included in the scope of protection of the present disclosure.

It should be noted that terms such as “first”, “second”, etc. in the description and claims and the above drawings of the present disclosure are used for distinguishing similar objects, and are not necessarily used for describing a specific order or a precedence order. It should be understood that data described in such way may be exchanged in appropriate conditions, so that the embodiments of the present disclosure described herein may be implemented in a different order besides the order graphically displayed or described. In addition, terms such as “comprise” and “have” and any other deformations thereof mean “including, but not limited to”, for example, a process, method, system, product or apparatus containing a series of steps or units is not necessarily limited to the clearly listed steps or units, but may include steps or units that are not clearly listed or that are inherent for the process, method, product or apparatus.

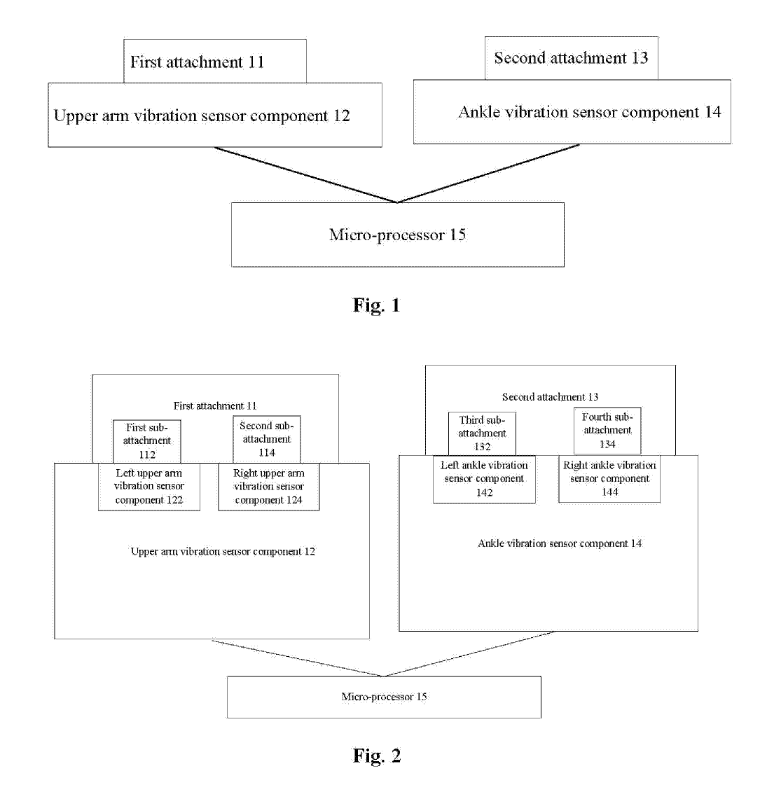

Tissue physiological activities have, for example, superficial arteries of human body, such as radial artery, brachial artery, carotid artery, subclavian artery and other areas. The artery blood vessels generally is very close to the skin surface; blood vessel vibrations caused by pulse waves may be directly transmitted and are reflected to the vibration of the adjacent skin surface. Lots of clinical measurement results show that synthetic signals (such as the form (wave shape), intensity (wave amplitude), rate (wave rate), rhythm (wave period) and the like expressed by pulse waves reflect many physiological and pathological characteristics of cardiovascular systems to certain extent. In relevant art, various discomforts exist when a cuff device is employed to measure arteriosclerosis; in the embodiment of the present disclosure, an arteriosclerosis measurement instrument is provided, FIG. 1 is a structural schematic diagram of an arteriosclerosis measurement instrument according to the embodiment of the present disclosure. As shown in FIG. 1, the arteriosclerosis measurement instrument 10 includes:
an upper arm vibration sensor component 12, attached onto a skin surface of a to-be-measured upper arm through a first attachment 11, wherein the upper arm vibration sensor component 12 is configured to collect an upper-arm pulse vibration signal of the to-be-measured upper arm;
an ankle vibration sensor component 14, attached onto a skin surface of a to-be-measured ankle through a second attachment 13, wherein the ankle vibration sensor component 14 is configured to collect an ankle pulse vibration signal of the to-be-measured ankle; and
a micro-processor 15, configured to determine a time difference between the above pulse vibration signals of a tested person according to the upper-arm pulse vibration signal collected by the upper arm vibration sensor component 12 and the ankle pulse vibration signal collected by the ankle vibration sensor component 14, and configured to obtain an arteriosclerosis measurement result according to the determined transmission time difference. In should be noted that the transmission time difference is used for obtaining a baPWV; and according to the baPWV measurement result, an arteriosclerosis degree is indicated.

Through the above structure, the vibration sensor component is attached onto the skin surface to be measured through the attachment, the pulse vibration signal is collected through the vibration sensor component, and the arteriosclerosis measurement result is obtained according to the collected pulse vibration signal. Compared with the compressing of an inflatable cuff exerted onto the skin to be measured, in the adoption of attaching the vibration sensor component onto the to-be-measured skin surface through the attachment, the attaching force of the attachment is significantly less than the compressing force of the cuff, the comfort is obviously enhanced, and many diseases inapplicable to compressing may be measured. In this way, the arteriosclerosis measurement result is successfully obtained in a condition of not compressing the skin surface to be measured; thus, the technical problem of insufficient universality when an inflatable cuff device is employed to measure arteriosclerosis is solved.

Pulse vibration not only may be viewed as the vibration of an artery blood vessel in vivo, but also may be viewed as the vibration of a skin surface caused by the blood vessel. Considering that it is extremely complicated to directly detect the vibration of an artery blood vessel in vivo and that the accuracy thereof is low. In order to operate conveniently and improve the accuracy of the measurement result, by reference to a standard arteriosclerosis measurement method, a preferred arteriosclerosis measurement instrument is provided in this embodiment. FIG. 2 is a preferred structural schematic diagram (1) of an arteriosclerosis measurement instrument according to the embodiment of the present disclosure. As shown in FIG. 2, the upper arm vibration sensor component 12 may include: a left upper arm vibration sensor component 122 attached onto a skin surface of a to-be-measured left upper arm through a first sub-attachment 112, and/or a right upper arm vibration sensor component 124 attached onto a skin surface of a to-be-measured right upper arm through a second sub-attachment 114, wherein the first attachment 11 includes the first sub-attachment 112 and/or the second sub-attachment 114; the ankle vibration sensor component 14 may include: a left ankle vibration sensor component 142 attached onto a skin surface of a to-be-measured left ankle through a third sub-attachment 132, and/or a right ankle vibration sensor component 144 attached onto a skin surface of a to-be-measured right ankle through a fourth sub-attachment 134, wherein the second attachment 13 includes the third sub-attachment 132 and/or the fourth sub-attachment 134. Through the measurement for the arms and legs of a tested body, the accuracy and normalization of the arteriosclerosis measurement result are improved.

In order to collect the upper-arm pulse vibration signal and the ankle pulse vibration signal, the upper arm vibration sensor component 12 and the ankle vibration sensor component 14 may work through many ways, for example, the upper arm vibration sensor component 12 may include: an upper arm Radio Frequency (RF) antenna component and an upper arm sensor (not shown in the drawings), wherein the upper arm RF antenna component is configured to excite an alternating electric field according to an input RF signal, receive an upper arm electrical field disturbance signal, and transmit the received upper arm electrical field disturbance signal to the upper arm sensor, wherein the upper arm electrical field disturbance signal may be an upper arm amplitude-modulated signal which is generated by modulating an amplitude of the upper arm pulse vibration signal of the skin surface of the to-be-measured upper arm onto the alternating electric field, when the alternating electric field is disturbed by the upper arm pulse vibration signal of the skin surface of the to-be-measured upper arm; the upper arm sensor is configured to receive the upper arm electrical field disturbance signal transmitted by the upper arm RF antenna, demodulate out the upper arm pulse vibration signal from the upper arm electrical field disturbance signal, and transmit the demodulated upper arm pulse vibration signal to the micro-processor 15.

Another example, the ankle vibration sensor component 14 may include: an ankle RF antenna component and ankle sensor (not shown in the drawings), wherein the ankle RF antenna component is configured to excite an alternating electric field according to an input RF signal, receive an ankle electrical field disturbance signal, and transmit the received ankle electrical field disturbance signal to the ankle sensor, wherein the ankle electrical field disturbance signal may be an ankle amplitude-modulated signal which is generated by modulating an amplitude of the ankle pulse vibration signal of the skin surface of the to-be-measured ankle onto the alternating electric field, when the alternating electric field is disturbed by the ankle pulse vibration signal of the skin surface of the to-be-measured ankle; the ankle sensor is further configured to receive the ankle electrical field disturbance signal transmitted by the ankle RF antenna, demodulate out the ankle pulse vibration signal from the ankle electrical field disturbance signal, and transmit the demodulated ankle pulse vibration signal to the micro-processor 15.

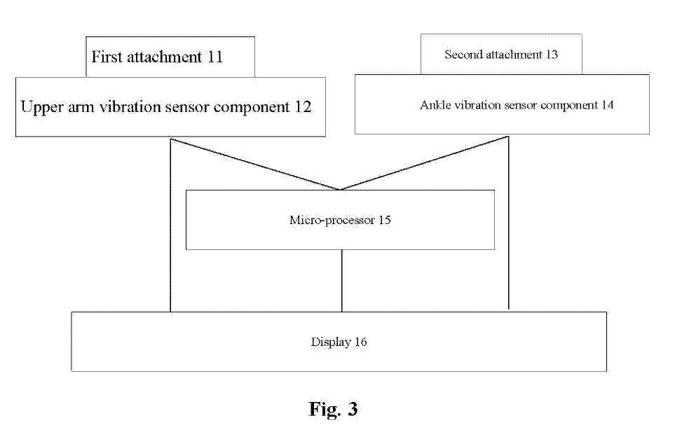

To make the measurement result more visual or the process result in the measurement process more clear, the arteriosclerosis measurement instrument may further include: a display 16. FIG. 3 is a preferred structure block diagram (2) of an arteriosclerosis measurement instrument according to the embodiment of the present disclosure. As shown in FIG. 3, the arteriosclerosis measurement instrument further includes a display 16, besides all the structure shown in FIG. 1, with specific description as below.

The display 16 is configured to display at least one of the following: the upper-arm pulse vibration signal collected by the upper arm vibration sensor component 12, the ankle pulse vibration signal collected by the ankle vibration sensor component 15 and the determined arteriosclerosis measurement result.

It should be noted that the first attachment and the second attachment may be of many types, merely if one surface thereof is capable of being stuck to the to-be-measured skin surface conveniently, while the other surface is capable of being stuck to the vibration sensor component conveniently, for example, the first attachment may be a sticking assembly or an adsorbing assembly, and/or, the second attachment may be a sticking assembly or an adsorbing assembly, wherein the sticking assembly may be a double faced adhesive tape, and/or, the adsorbing assembly may be a suction cup, wherein the suction cup is a kind of sheet having an adsorption function. The material of the suction cup may be of many types, for example, plastic or resin, etc.

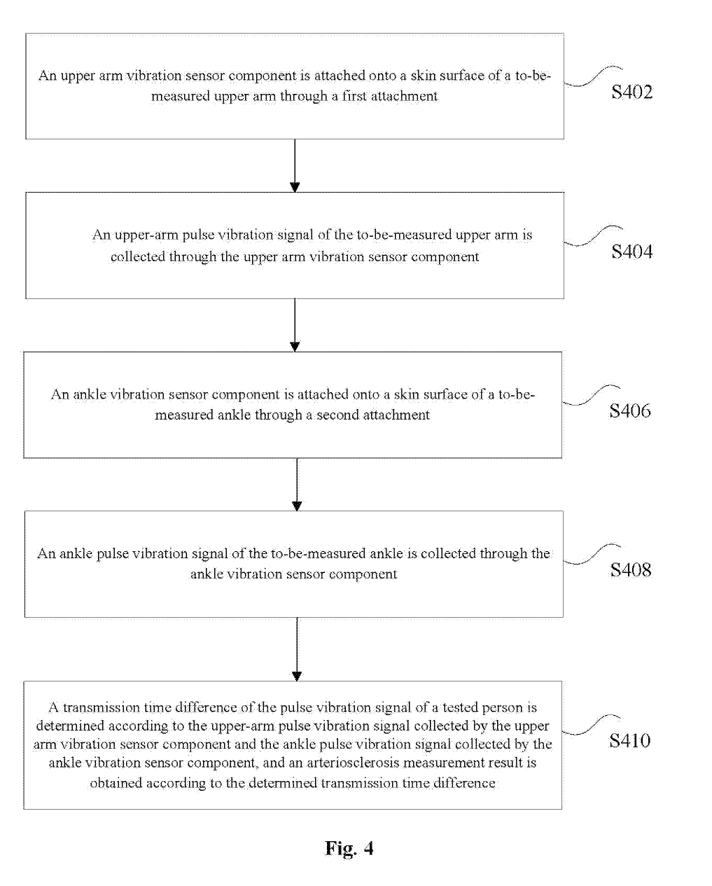

FIG. 4 is a flowchart of an arteriosclerosis measurement method according to the embodiment of the present disclosure. As shown in FIG. 4, the arteriosclerosis measurement method includes the following steps:

In S402, an upper arm vibration sensor component is attached onto a skin surface of a to-be-measured upper arm through a first attachment.

In S404, an upper-arm pulse vibration signal of the to-be-measured upper arm is collected through the upper arm vibration sensor component.

In S406, an ankle vibration sensor component is attached onto the skin surface of a to-be-measured ankle through a second attachment.

In S408, an ankle pulse vibration signal of the to-be-measured ankle is collected through the ankle vibration sensor component.

In S410, the time difference between the pulse vibration signal of a tested person is determined according to the upper-arm pulse vibration signal collected by the upper arm vibration sensor component and the ankle pulse vibration signal collected by the ankle vibration sensor component, and an arteriosclerosis measurement result is obtained according to the determined time difference between arrival.

Through the above steps, the vibration sensor component is attached onto the skin surface to be measured through the attachment, the pulse vibration signal is collected through the vibration sensor component, and the arteriosclerosis measurement result is obtained according to the collected pulse vibration signal. In this way, the arteriosclerosis measurement result is successfully obtained in the condition of not compressing the skin surface to be measured; thus, the technical problem of insufficient universality when an inflatable cuff device is employed to measure arteriosclerosis is solved.

In conjunction with the structure of the above arteriosclerosis measurement instrument, optionally, the step that the upper arm vibration sensor component 12 is attached onto the skin surface of the to-be-measured upper arm through the first attachment 11 includes: a left upper arm vibration sensor component 122 is attached onto a skin surface of a to-be-measured left upper arm through a first sub-attachment 112, and/or a right upper arm vibration sensor component 124 is attached onto a skin surface of a to-be-measured right upper arm through a second sub-attachment 114, wherein the first attachment 11 includes the first sub-attachment 112 and/or the second sub-attachment 114, the upper arm vibration sensor component 12 includes the left upper arm vibration sensor component 122 and/or the right upper arm vibration sensor component 124; and the step that the ankle vibration sensor component 14 is attached onto the skin surface of the to-be-measured ankle through the second attachment 13 includes: a left ankle vibration sensor component 142 is attached onto a skin surface of a to-be-measured left ankle through a third sub-attachment 132, and/or a right ankle vibration sensor component 144 is attached onto a skin surface of a to-be-measured right ankle through a fourth sub-attachment 134, wherein the second attachment 13 includes the third sub-attachment 132 and/or the fourth sub-attachment 134, and the ankle vibration sensor component 14 includes the left ankle vibration sensor component 142 and/or the right ankle vibration sensor component 144.

Optionally, the upper arm vibration sensor component 12 includes: an upper arm RF antenna component and an upper arm sensor (not shown in the drawings); the step that an upper-arm pulse vibration signal of the to-be-measured upper arm is collected through the upper arm vibration sensor component 12 may include: the upper arm RF antenna component may excite an alternating electric field according to an input RF signal, an upper arm electrical field disturbance signal is received through the upper arm RF antenna component, and the upper arm sensor demodulates out the upper arm pulse vibration signal from the upper arm electrical field disturbance signal; wherein the upper arm electrical field disturbance signal is an upper arm amplitude-modulated signal which is generated by modulating an amplitude of the upper arm pulse vibration signal of the skin surface of the to-be-measured arm onto the alternating electric field, when the alternating electric field is disturbed by the upper arm pulse vibration signal of the skin surface of the to-be-measured upper arm.

The ankle vibration sensor component 14 may include: an ankle RF antenna component and an ankle sensor (not shown in the drawings); the step that an ankle pulse vibration signal of the to-be-measured ankle is collected through the ankle vibration sensor component 14 includes: the ankle RF antenna component excites an alternating electric field according to an input RF signal, an ankle electrical field disturbance signal is received through the ankle RF antenna component, and the ankle sensor demodulates out the ankle pulse vibration signal from the ankle electrical field disturbance signal; wherein the ankle electrical field disturbance signal is an ankle amplitude-modulated signal which is generated by modulating an amplitude of the ankle pulse vibration signal of the skin surface of the to-be-measured ankle onto the alternating electric field, when the alternating electric field is disturbed by the ankle pulse vibration signal of the skin surface of the to-be-measured ankle.

To further improve the accuracy of the measurement result, the step that the upper arm vibration sensor component 12 is attached onto the skin surface of the to-be-measured upper arm through the first attachment 11 includes: an upper arm pulse sensitive part of the skin surface of the to-be-measured upper arm is sensed firstly, then, the upper arm vibration sensor component 12 is attached onto the upper arm pulse sensitive part of the skin surface of the to-be-measured upper arm through the first attachment; and/or, the step that the ankle vibration sensor component 13 is attached onto the skin surface of the to-be-measured ankle through the second attachment 13 includes: an ankle pulse sensitive part of the skin surface of the to-be-measured ankle is sensed, and the ankle vibration sensor component 14 is attached onto the ankle pulse sensitive part of the skin surface of the to-be-measured ankle through the second attachment 13.

Optionally, after an upper-arm pulse vibration signal of the to-be-measured upper arm is collected through the upper arm vibration sensor component 12, and/or, an ankle pulse vibration signal of the to-be-measured ankle is collected through the ankle vibration sensor component 14, the method further includes: the collected upper-arm pulse vibration signal and the collected ankle pulse vibration signal are displayed; or, after an arteriosclerosis measurement result is obtained according to the determined transmission time difference, the method further includes: the determined arteriosclerosis measurement result is displayed. Through the display of the measurement result or the display of various information in the measurement process, the measurement is more visual and accurate.

Optionally, the first attachment may be of many types, for example, a sticking assembly or an adsorbing assembly, and/or, the second attachment may be of many types, for example, a sticking assembly or an adsorbing assembly.

Herein, the sticking assembly may be a double faced adhesive tape, and/or, the adsorbing assembly may be a suction cup, wherein the suction cup is a kind of sheet having an adsorption function. The material of the suction cup may be of many types, for example, plastic or resin, etc.

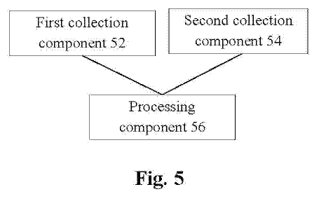

In the embodiment of the present disclosure, an arteriosclerosis measurement device may be further provided. FIG. 5 is a structure block diagram of an arteriosclerosis measurement device according to the embodiment of the present disclosure. As shown in FIG. 5, the device includes: a first collection component 52, a second collection component 54 and a processing component 56. The device is described below.

The first collection component 52 is configured to: after an upper arm vibration sensor component is attached onto a skin surface of a to-be-measured upper arm through a first attachment 11, collect an upper-arm pulse vibration signal of the to-be-measured upper arm through an upper arm vibration sensor component 12.

The second collection component 54 is configured to: after an ankle vibration sensor component 14 is attached onto a skin surface of a to-be-measured ankle through a second attachment 13, collect an ankle pulse vibration signal of a to-be-measured ankle through an ankle vibration sensor component 14.

The processing component 56 is connected to the first collection component 52 and the second collection component 54, and is configured to: determine a time difference between the above pulse vibration signals of a tested person according to the upper-arm pulse vibration signal collected by the upper arm vibration sensor component 12 and the ankle pulse vibration signal collected by the ankle vibration sensor component 14, and obtain an arteriosclerosis measurement result according to the determined transmission time difference.

In the embodiment of the present disclosure, a storage medium is further provided, which includes a stored program, wherein, when running, the program controls the equipment on which the storage medium is located to execute any one arteriosclerosis measurement method describe above.

In the embodiment of the present disclosure, a processor is further provided, which is configured to run a program, wherein, when running, the program executes any one arteriosclerosis measurement method described above.

In the embodiment of the present disclosure, a terminal is further provided, including: a processor and a display, wherein
the processor runs a program, wherein, when running, the program executes the processing steps included in any one arteriosclerosis measurement method described above; and the display is configured to display the processed result and/or the process result of the processor.

The sequence number of the above embodiments of the present disclosure is merely for description, not representing the superiority and inferiority of the embodiments.

In the above embodiments of the present disclosure, the description of each embodiment has a respective emphasis, what not described in certain embodiment may refer to relevant description in other embodiments.

In the embodiments provided in this application, it should be understood that the disclosed technical content may be realized through other ways. The device embodiments described above are exemplary only, for example, the division of unit is a division of logical function merely, and may select other division methods during actual implementation, for example, a plurality of units or components may be combined, or may be integrated into another system, or some feature may be neglected or not executed. In addition, the mutual coupling, or direct coupling, or communication connection between the displayed or discussed components may be realized through some interfaces; the indirect coupling or communication connection between devices or units may be electrically, mechanically or in other forms.

The above unit described as a separate component may be or may not be physically separated; the component, displayed as a unit, may be or may not be a physical unit, that is, it may be located at one place, or may be distributed on a plurality of network units. Part or all units may be selected to realize the purpose of the embodiment scheme according to actual needs.

In addition, each function unit in each embodiment of the present disclosure may be integrated in one processing unit, or each unit exists physically separately, or two or more units are integrated in one unit. The above integrated units may be realized in the form of hardware, or in the form of software function units.

When the integrated units are realized in the form of software function units and are sold or used as an independent product, they may be stored in computer readable storage medium. Based on this understanding, the technical scheme of the present disclosure or the part making a contribution to the existing technology or the entirety or part of the technical scheme on essence may be embodied in the form of software product. This computer software product is stored in a storage medium, including a number of instructions that enables a computer device (which might be a computer, a server or a network device, etc.) to execute part or the entirety of the method described in each embodiment of the present disclosure. The aforementioned storage medium includes: USB flash disk, Read-Only Memory (ROM), Random Access Memory (RAM), mobile hard disk, diskette or compact disc and various mediums capable of storing program codes.

The above are the preferred implementations of the present disclosure merely. It should be noted that, for the ordinary staff in this art, various improvements and modifications may be made without departing from the principle of the present disclosure, and these improvements and modifications shall fall into the scope of protection of the present disclosure.

What is claimed is:

1. An arteriosclerosis measurement instrument comprising:
an upper arm vibration sensor component attached onto a skin surface of a to-be-measured upper arm through a first attachment, wherein the upper arm vibration sensor component is configured to collect an upper-arm pulse vibration signal of the to-be-measured upper arm;
an ankle vibration sensor component attached onto a skin surface of a to-be-measured ankle through a second attachment, wherein the ankle vibration sensor component is configured to collect an ankle pulse vibration signal of the to-be-measured ankle; and
a micro-processor, configured to determine a lime difference between the above pulse vibration signals of a tested person according to the upper-arm pulse vibration signal collected by the upper arm vibration sensor component and the ankle pulse vibration signal collected by the ankle vibration sensor component, and configured to obtain an arteriosclerosis measurement result according to the determined transmission time difference.

2. The arteriosclerosis measurement instrument as claimed in claim 1, wherein
the upper arm vibration sensor component comprises: a left upper arm vibration sensor component attached onto a skin surface of a to-be-measured left upper arm through a first sub-attachment, and/or a right upper arm vibration sensor component attached onto a skin surface of a to-be-measured right upper arm through a second sub-attachment, wherein the first attachment comprises the first sub-attachment and/or the second sub-attachment; and
the ankle vibration sensor component comprises: a left ankle vibration sensor component attached onto a skin Surface of a lo-he-measured left ankle through a third sub-attachment, and/or a right ankle vibration sensor component attached onto a skin surface of a to-be-measured right ankle through a fourth sub-attachment, wherein the second attachment comprises the third sub-attachment and/or the fourth sub-attachment.

3. The arteriosclerosis measurement instrument as claimed in claim 1, further comprising:
a display, configured to display at least one of following: the upper-arm, pulse vibration signal collected by the upper arm vibration sensor component, the ankle pulse vibration signal collected by the ankle vibration sensor component and the determined arteriosclerosis measurement result.

4. The arteriosclerosis measurement instrument as claimed in claim 1, wherein
the first attachment is a sticking assembly or an adsorbing assembly, and/or, the second attachment is a sticking assembly or an adsorbing assembly.

5. The arteriosclerosis measurement instrument as claimed in claim 4, wherein
the slicking assembly is a double faced adhesive tape, and/or, the adsorbing assembly is a suction cup.

6. An arteriosclerosis measurement method, comprising:
attaching an upper arm vibration sensor component onto a skin surface of a to-be-measured upper arm through a first attachment;
collecting an upper-arm pulse vibration signal of the to-be-measured upper arm through the upper arm vibration sensor component;
attaching an ankle vibration sensor component onto a skin surface of a to-be-measured ankle through a second attachment;
collecting an ankle pulse vibration signal of the to-be-measured ankle through the ankle vibration sensor component;
determining a time difference between the above pulse vibration signals of a tested person according to the upper-arm pulse vibration signal collected by the upper arm vibration sensor component and the ankle pulse vibration signal collected by the ankle vibration sensor component, and obtaining an arteriosclerosis measurement result according to the determined transmission lime difference.

7. The method as claimed in claim 6, wherein
attaching the upper arm vibration sensor component onto the skin surface of the to-be-measured upper arm through the first attachment comprises: attaching a left upper arm vibration sensor component onto a skin surface of a to-be-measured left upper arm through a first sub-attachment, and/or attaching a right upper arm vibration sensor component onto a skin surface of a to-be-measured right upper arm through a second sub-attachment, wherein the first attachment comprises the first sub-attachment and/or the second sub-attachment, the upper arm vibration sensor component comprises the left upper arm vibration sensor component and/or the right upper arm vibration sensor component; and
attaching the ankle vibration sensor component onto the skin surface of the to-be-measured ankle through the second attachment comprises: attaching a left ankle vibration sensor component onto a skin surface of a to-be-measured left ankle through a third sub-attachment, and/or attaching a right ankle vibration sensor component onto a skin surface of a to-be-measured right ankle through a fourth sub-attachment, wherein the second attachment comprises the third sub-attachment and/or the fourth sub-attachment, and the ankle vibration sensor component comprises the left ankle vibration sensor component and/or the right ankle vibration sensor component.

8. The method as claimed in claim 6, wherein
attaching the upper arm vibration sensor component onto the skin surface of the to-be-measured upper arm through the first attachment comprises: sensing an upper arm pulse sensitive part of the skin surface of the to-be-measured upper arm, and attaching the upper arm vibration sensor component onto the upper arm pulse sensitive part of the skin surface of the to-be-measured upper arm through the first attachment;
and/or,
attaching the ankle vibration sensor component onto the skin surface of the to-be-measured ankle through the second attachment comprises: sensing an ankle pulse sensitive part of the skin surface of the to-be-measured ankle, and attaching the ankle vibration sensor component onto the ankle pulse sensitive part of the skin surface of the to-be-measured ankle through the second attachment.

9. The method as claimed in claim 6, wherein
after collecting the upper-arm pulse vibration signal of the to-be-measured upper arm through the upper arm vibration sensor component, and/or, collecting the ankle pulse vibration signal of the to-be-measured ankle through the ankle vibration sensor component, the method further comprises: displaying the collected upper-arm pulse vibration signal and the collected ankle pulse vibration signal;
or,
after obtaining the arteriosclerosis measurement result according to the determined transmission time difference, the method further comprises: displaying the determined arteriosclerosis measurement result.

10. The method as claimed in claim 6, wherein
the first attachment is a sticking assembly or an adsorbing assembly, and/or, the second attachment is a sticking assembly or an adsorbing assembly.

11. The method as claimed in claim 10, wherein
the sticking assembly is a double faced adhesive tape, and/or, the adsorbing assembly is a suction cup.

12. The arteriosclerosis measurement instrument as claimed claim 2, wherein
the first attachment is a sticking assembly or an adsorbing assembly, and/or, the second attachment is a sticking assembly or an adsorbing assembly.

13. The arteriosclerosis measurement instrument as claimed claim 3, wherein
the first attachment is a sticking assembly or an adsorbing assembly, and/or, the second attachment is a sticking assembly or an adsorbing assembly.

14. The arteriosclerosis measurement instrument as claimed claim 12, wherein
the sticking assembly is a double faced adhesive tape, and/or, the adsorbing assembly is a suction cup.

15. The arteriosclerosis measurement instrument as claimed in claim 13, wherein
the sticking assembly is a double faced adhesive tape, and/or, the absorbing assembly is a suction cup.

16. The arteriosclerosis measurement instrument as claimed in claim 1, wherein
the first attachment is a first restraining strap, wherein the first restraining strap is used for restraining the to-be-measured upper arm, an inner surface of the first restraining strap is attached onto the skin surface of the to-be-measure upper arm, and the upper arm vibration sensor component is fixed onto an outer surface of the first restraining strap by means of repeatedly disassembling and mounting;
the second attachment is a second restraining strap, wherein the second restraining strap is used for restraining the to-be-measured ankle, an inner surface of the second restraining strap is attached onto the skin surface of the to-be-measured ankle, and the ankle vibration sensor component is fixed onto an outer surface of the second restraining strap by means of repeatedly disassembling and mounting.

17. The arteriosclerosis measurement instrument as claimed in claim 16, wherein
a first window is disposed on a portion corresponding to a detection region of the upper arm vibration sensor component, wherein a first supporting structure is taken as an appendant to be disposed on a bottom, corresponding to the upper arm vibration sensor component, of the first window;
a second window is disposed on a portion corresponding to a detection region of the ankle vibration sensor component, wherein a second supporting structure is taken as an appendant to be disposed on a bottom, corresponding to ankle vibration sensor component, of the second window.

18. The method as claimed in claim 6, wherein
the first attachment is a first restraining strap, wherein the first restraining strap is used for restraining the to-be-measured upper arm, an inner surface of the first restraining strap is attached onto the skin surface of the to-be-measured upper arm, and the upper arm vibration sensor component is fixed onto an outer surface of the first restraining strap by means of repeatedly disassembling and mounting;
the second attachment is a second restraining strap, wherein the second restraining strap is used for restraining the to-be-measured ankle, an inner surface of the second restraining strap is attached onto the skin surface of the to-be-measured ankle, and the ankle vibration sensor component is fixed onto an outer surface of the second restraining strap by means of repeatedly disassembling and mounting.

19. The method as claimed in claim 18, wherein
a first window is disposed on a portion corresponding to a detection region of the upper arm vibration sensor component, wherein a first supporting structure is taken as an appendant to be disposed on a bottom, corresponding to the upper arm vibration sensor component, of the first window;
a second window is disposed on a portion corresponding to a detection region of the ankle vibration sensor component, wherein a second supporting structure is taken as an appendant to be disposed on a bottom, corresponding to ankle vibration sensor component, of the second window.

20. The method as claimed in claim 7, wherein
the first attachment is a sticking assembly or an absorbing assembly, and/or, the second attachment is a sticking assembly or an absorbing assembly.
